(19) United States
(12) Patent Application Publication
(10) Pub. No.: US 2023/0260662 A1
(43) Pub. Date: Aug. 17, 2023

(54) GENERATIVE MANIFOLD NETWORKS FOR PREDICTION AND SIMULATION OF COMPLEX SYSTEMS

(71) Applicant: THE SALK INSTITUTE FOR BIOLOGICAL STUIDES, La Jolla, CA (US)

(72) Inventors: Gerald PAO, La Jolla, CA (US); Cameron SMITH, La Jolla, CA (US)

(73) Assignee: THE SALK INSTITUTE FOR BIOLOGICAL STUIDES, La Jolla, CA (US)

(21) Appl. No.: 18/003,005

(22) PCT Filed: Jun. 26, 2021
(86) PCT No.: PCT/US2021/039271

§ 371 Date: Dec. 22, 2022

Related U.S. Application Data
(60) Provisional application No. 63/044,603, filed on Jun. 26, 2020.

(51) Int. Cl. G16H 50/50; G06F 17/15

(52) U.S. Cl. CPC G16H 50/50; G06F 17/153

(57) ABSTRACT

Disclosed herein are generative manifold networks (GMNs) which enable accurate prediction and modeling of complex interconnected systems.

### CROSS-REFERENCE

The present application claims priority to U.S. Provisional Patent Application No. 63/044,603, filed on Jun. 26, 2020, entitled “GENERATIVE MANIFOLD NETWORKS FOR PREDICTION AND SIMULATION OF COMPLEX SYSTEMS,” which is herein incorporated by reference in its entirety for all purposes.

### TECHNICAL FIELD

The subject matter described herein relates to systems and methods for network-based prediction and simulation of complex systems. Such systems and methods, which may be referred to as generative manifold networks, may have particular but not exclusive utility for simulation of neural activity.

### BACKGROUND

Brain activity is a highly nonlinear phenomenon, and as such, dynamical relationships between neurons and expressed behaviors have been described on low dimensional attractor manifolds. Neuroscience has a long history of studying oscillations and intermittent activity that may be compatible with descriptions of neural activity as dynamics on an attractor. In recent years an accumulating body of evidence shows that a large number of neuronal activity dynamics are well described in terms of low dimensional attractor manifolds. Examples in the literature find these low dimensional manifolds within the ensemble properties of relatively large populations of neurons.

The presence of coherent dynamics in neural activity has been recognized for at least two decades in one form of another, however much excitement has been generated in the last few years upon discovering how ubiquitous these dynamics are. These early manifolds, however appealing, were observations of coherent behavior without explicit dynamic information.

Thanks to recent developments in imaging technology, it is now possible and increasingly common to obtain neural activity datasets on whole brains from experimental model organisms. This type of data, when coupled with simultaneous quantitative behaviors, allows study of possible mapping functions between neural activity and behavior.

The idea of capturing an organism's brain activity and reproducing its activity in a computer (e.g., to guide the actions of a real robot, simulated robot, or simulated body) has been a longstanding idea but has been previously considered unachievable due to a large number of technical hurdles, as well as gaps in human understanding of basic neuroscience. For example, downloading a representation of a brain into a robot, or using a brain to control a robot, have previously been considered impossible due to limited understanding of the way brains work.

The information included in this Background section of the specification, including any references cited herein and any description or discussion thereof, is included for technical reference purposes only and is not to be regarded as subject matter by which the scope of the disclosure is to be bound.

### SUMMARY

Disclosed herein are systems and methods for network-based prediction and simulation of complex systems. Such systems and methods may be referred to herein as generative manifold networks (GMNs). The GMNs disclosed herein may have particular, but not exclusive, utility for prediction of neural activity. However, the GMNs may be applicable to any nonlinear network, and so may have wide ranging applications in other areas. GMNs can be applied to any simulations to explore possible scenarios for long-term planning of complex systems, where the system is too complex to be captured by equations for which there is sufficient timeseries data. In addition, GMNs can be used for short-term predictions of these complex systems, including systems that have large amounts of data. Examples include, but are not limited to, brain activity, internet traffic, highway traffic, interconnected citywide activities, weather, stock market, commodities markets, hedge funds, investment banks, online behavior, etc. In addition, GMNs can be used to download representations of a real or simulated brain into a real or simulated robot.

In a non-limiting example, a GMN begins with simultaneous time series recordings of near-whole-brain neural activity and movement (walking) in transgenic Drosophila melanogaster. From these data, a model is constructed. The model can generate walking behaviors in the plane that are not only similar to those observed in the real fly, but that also contain realistic observed fly movement behavior that was not used in model training Among several strong validations, the GMNs most surprisingly produces artificial generated time series that exhibit realistic fly resting state behaviors that were not present in the training data. This suggests that this type of network may embody emergent properties hidden in the brain that are not explicitly represented in the training set used to build the model.

An example GMN is able to make short-term predictions and perform realistic, high-quality simulations over long periods of time. GMNs, as disclosed herein, can be expanded for use in large systems, and can incorporate features to make it capable of learning on its own, thus embodying a new type of artificial intelligence (AI) that takes advantage of what real brains do, but is not limited to such behaviors.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the GMNs, as defined in the claims, is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 shows a simplified diagram of a method for training a generative manifold network (GMN), in accordance with various embodiments of the present disclosure.

FIG. 2 is a simplified diagram of a method for using a GMN, in accordance with various embodiments of the present disclosure.

FIG. 3 is a block diagram of a computer-based system for determining a kinetic parameter, in accordance with various embodiments of the present disclosure.

FIG. 4 is a block diagram of a computer system, in accordance with various embodiments of the present disclosure.

FIG. 5 is a simplified diagram of training and using a GMN, in accordance with various embodiments of the present disclosure.

FIG. 6 shows examples of extraction of relationships between variables of a given generative manifold network (GMN) and simulation outputs of the GMN, in accordance with various embodiments of the present disclosure.

FIG. 7 shows illustrative inputs and outputs of an example GMN, in accordance with various embodiments of the present disclosure.

FIG. 8 shows a comparison between real and generated movements, in accordance with various embodiments of the present disclosure.

FIG. 9 shows a comparison of real fly movements with movements generated by different modeling approaches, in accordance with various embodiments of the present disclosure.

FIG. 10 shows a validation scheme of the GMN generated data, in accordance with various embodiments of the present disclosure.

FIG. 11 shows the prediction skill of GMNs compared to three other methods, to validate similarity to real motion data, in accordance with various embodiments of the present disclosure.

FIG. 12 shows an example of generating a model of neural activity and behavior of an organism for download into a robot, in accordance with various embodiments of the present disclosure.

### DETAILED DESCRIPTION

In accordance with various embodiments of the present disclosure, a generative manifold network (GMN) system is provided which enables accurate prediction and modeling of complex interconnected systems.

Through dimensionality reduction approaches, most notably principal component analysis, one commonly finds that the low-dimensional embeddings of neural activity contain surprisingly coherent structure. It is in these low dimensional embeddings derived from larger populations, where most observations have been made that show remarkable coherence when projected into low-dimensional space on the surfaces of manifolds. The present disclosure provides a data-driven approach, based on networks of manifolds, that transforms empirical observations of neuronal activity into a realistic generative model.

This approach was demonstrated and validated on publicly available data, consisting of simultaneous time series recordings of near-whole-brain neural activity and movement (walking) in transgenic Drosophila melanogaster. From these data, a model was created that can generate walking behaviors in the plane that are not only similar to those observed in the real fly, but that also contain realistic observed fly movement behavior that was not used in model training Among several strong validations, the generative model most surprisingly produces artificial generated time series that exhibit realistic fly resting state behaviors that were not present in the training data. This suggests that this type of network may embody emergent properties hidden in the brain that are not explicitly represented in the training set used to build the model.

More recently, the view of attractor dynamics on manifold surfaces has started to deliver predictive power. Variability in neural activity, that was commonly thought to be noise, was shown to be largely accounted for by the trajectory deviations due the existence of additional dimensions on the surface of the manifold when compared to the arena in which the subjects (e.g., rats) were moving.

Methods have also been developed that generate naturalistic behaviors from artificial neural networks using reservoir computing or liquid state machine approaches. Generally, reservoir computing approaches have been extraordinarily successful in the prediction of low-dimensional chaotic systems. Their predictions even beyond their prediction horizons preserve the ergodic properties of the system and are realistic surrogates of the original analytically derived behaviors. However, these are finely tuned for particular systems, and so far have only been implementable in equation-based models. It is not clear at this point if these are adequate for empirically derived observations.

To predict movements of an organism using whole-brain activity may require a dataset that has simultaneous recordings of brain activity as well as the recorded motions of the organism. Currently, given experimental limitations, this is only possible with a few experimental species of animals. The disclosures herein utilized a dataset of from a headfixed Drosophila melanogaster on a Styrofoam ball, where whole brain activity was recorded using light field microscopy of signals derived from a transgenic fluorescent calcium indicator GCaMP6f, and walking motions were recorded from the rotation of the Styrofoam ball. The whole-brain dataset did not achieve single-neuron resolution, so the whole-brain activity was segmented using independent component analysis to identify 80 different areas in the whole brain that were deemed to have distinct activities. These time series, together with the recorded left-right and forward speed of motion of the flies on the Styrofoam ball, comprise our data. At this stage, the data were split into training and testing sets.

The present disclosure aids substantially in modeling of complex systems, by improving the fidelity of models without adding undue complexity. Implemented on a processor supplied with a training data set, the generative manifold network system disclosed herein provides practical prediction and modeling of the behavior of complex systems, including living brains. This improved modeling capability transforms task-specific neural networks into systems capable in principle of representing an entire brain, without the normally routine need to model individual biological neurons and their interconnections. This unconventional approach improves the functioning of complex system models.

The GMNs may be implemented as a method viewable on a display or downloadable into a robot, and operated by a control process executing on a processor that accepts inputs from a keyboard, mouse, touchscreen interface, and that is in communication with one or more sensors. In that regard, the control process performs certain specific operations in response to different inputs or selections made at different times.

These descriptions are provided for exemplary purposes only, and should not be considered to limit the scope of the generative manifold network system. Certain features may be added, removed, or modified without departing from the spirit of the claimed subject matter.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

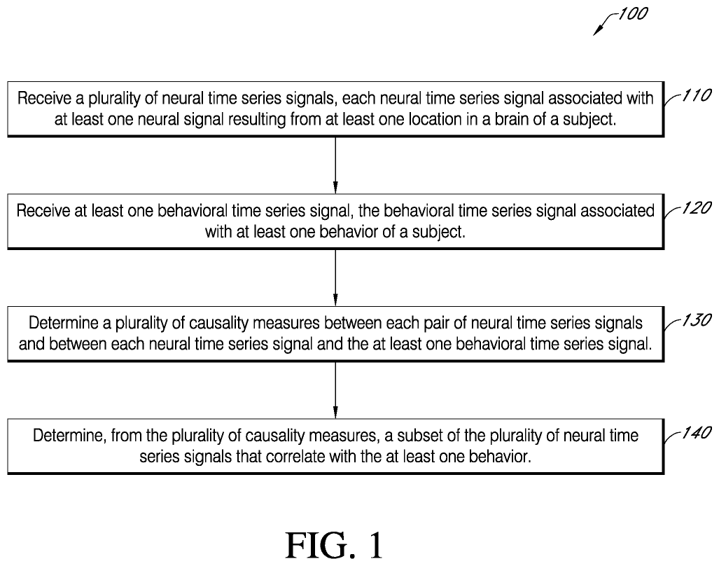

FIG. 1 shows a simplified diagram of a method 100 for training a GMN, in accordance with various embodiments of the present disclosure. In various embodiments, the method 100 comprises a first step 110 of receiving a plurality of neural time series signals. Each neural time series signal may be associated with at least one neural resulting from at least one location in a brain of a subject. A subject may be a human, non-human primate, chimpanzee, horse, dog, cat, rabbit, mouse, rat, fish, zebrafish, fly, or other subject. A neural time series signal may comprise an evoked potential signal, a far-field evoked potential signal, a near-field evoked potential signal, a single-neuron extracellular signal, a multi-neuron extracellular signal, a microelectrode signal, a microelectrode array signal, a sharp electrode signal, a patch-clamp electrode signal, an optical signal, a fluorescence signal, an intrinsic optical change signal, an electroencephalography (EEG) signal, a magnetoencephalography (MEG) signal, a magnetic resonance imaging (MRI) signal, or a functional MRI (fMRI) signal. The plurality of neural time series signals may comprise at least about 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60, 000, 70,000, 80,000, 90,000, 100,000, 200,000, 300,000, 400,000, 500,000, 600,000, 700,000, 800,000, 900,000, 1,000,000, or more neural time series signals. The plurality of neural time series signals may comprise at most about 1,000,000, 900,000, 800,000, 700,000, 600,000, 500,000, 400,000, 300,000, 200,000, 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 3, or 2 neural time series signals. The plurality of neural time series signals may comprise a number of neural time series signals that is within a range defined by any two of the preceding values.

In various embodiments, the method 100 comprises a second step 120 of receiving at least one behavioral time series signal. The behavioral time series signal may be associated with at least one behavior of the subject. The at least one behavioral time series signal may comprise a plurality of behavioral time series signals. The plurality of behavioral time series signals may comprise at least about 2, 3, 4, 5, 6, 7, 8, 9, 10, 20, 30, 40, 50, 60, 70, 80, 90, 100, 200, 300, 400, 500, 600, 700, 800, 900, 1,000, or more behavioral time series signals. The plurality of behavioral time series signals may comprise at most about 1,000, 900, 800, 700, 600, 500, 400, 300, 200, 100, 90, 80, 70, 60, 50, 40, 30, 20, 10, 9, 8, 7, 6, 5, 4, 3, or 2 behavioral time series signals.

In various embodiments, the method 100 comprises a third step 130 of determining a plurality of causality measures. The plurality of causality measures may be between each pair of neural time series signals and between each neural time series signal and the at least one behavioral time series signal. The plurality of causality measures may comprise at least about 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60, 000, 70,000, 80,000, 90,000, 100,000, 200,000, 300,000, 400,000, 500,000, 600,000, 700,000, 800,000, 900,000, 1,000,000, or more causality measures. The plurality of causality measures may comprise at most about 1,000,000, 900,000, 800,000, 700,000, 600,000, 500,000, 400,000, 300,000, 200,000, 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 3, or 2 causality measures. The plurality of causality measures may comprise a number of causality measures that is within a range defined by any two of the preceding values.

In various embodiments, the plurality of causality measures is determined by: (i) determining a mapping matrix between the plurality of neural time series signals and the at least one behavioral time signal; (ii) determining a correlation matrix between each pair of neural time series signals and between each neural time series signal and the at least one behavioral time signal; and (iii) subtracting the correlation matrix from the mapping matrix to thereby form a predictability matrix. The mapping matrix may comprise a convergent cross mapping (CCM) matrix, as described herein. The correlation matrix may comprise a linear correlation matrix, as described herein. The correlation matrix may comprise a Pearson correlation matrix, as described herein. The mapping matrix may be referred to herein as ρ_{ccm} and may have entries ρ_{ccm}_{ᵢⱼ}. The correlation matrix may be referred to herein as ρ_{corr} and may have entries ρ_{corr}_{ᵢⱼ}. The predictability matrix may be referred to herein as ρ_{diff} and may have entries ρ_{diff}_{ᵢⱼ}.

In various embodiments, the method 100 comprises a fourth step 140 of determining, from the plurality of causality measures, a subset of the plurality of neural time series signals that correlate with the at least one behavior. The subset may be determined by: (i) determining an acyclic directed graph based on the predictability matrix; and (ii) determining, from the acyclic directed graph, the subset. The acyclic directed graph may be referred to herein as G(ρ_{diff}).

In various embodiments, the method 100 comprises measuring the plurality of neural time series signals. The method 100 may comprise measuring the at least one behavioral time series signal.

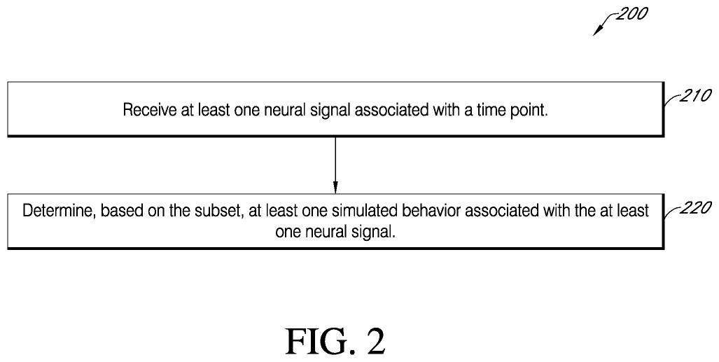

FIG. 2 is a simplified diagram of a method for using a GMN, in accordance with various embodiments of the present disclosure. In various embodiments, the method 200 comprises a first step 210 of receiving at least one neural signal associated with a time point.

In various embodiments, the method 200 comprises a second step 220 of determining, based on the subset, at least one simulated behavior associated with the at least one neural signal. The at least one simulated behavior may be determined by: (i) determining, based on the subset, a plurality of simulated neural signals associated with a plurality of simulated time points, each simulated time point occurring at a later time than the time point; (ii) determining a geometric weighting between the plurality of simulated neural signals; and (iii) determining, based on the geometric weighting, the at least one simulated behavior. The geometric weighting may comprise a simplex weighting. Operations (i)-(iii) may be repeated to determine a plurality of simulated behaviors. For instance, operations (i)-(iii) may be repeated at least about 1, 2, 3, 4, 5, 6, 7, 8, 9, 10, 20, 30, 40, 50, 60, 70, 80, 90, 100, or more times. Operations (i)-(iii) may be repeated at most about 100, 90, 80, 70, 60, 50, 40, 30, 20, 10, 9, 8, 7, 6, 5, 4, 3, 2, or 1 times. Operations (i)-(iii) may be repeated a number of times that is within a range defined by any two of the preceding values.

In accordance with various embodiments, the method 200 comprises measuring the at least one neural signal associated with the time point.

In accordance with various embodiments, the methods 100 and 200 may be combined.

In various embodiments, at least a portion of the methods for estimating a kinetic parameter can be implemented via software, hardware, firmware, or a combination thereof.

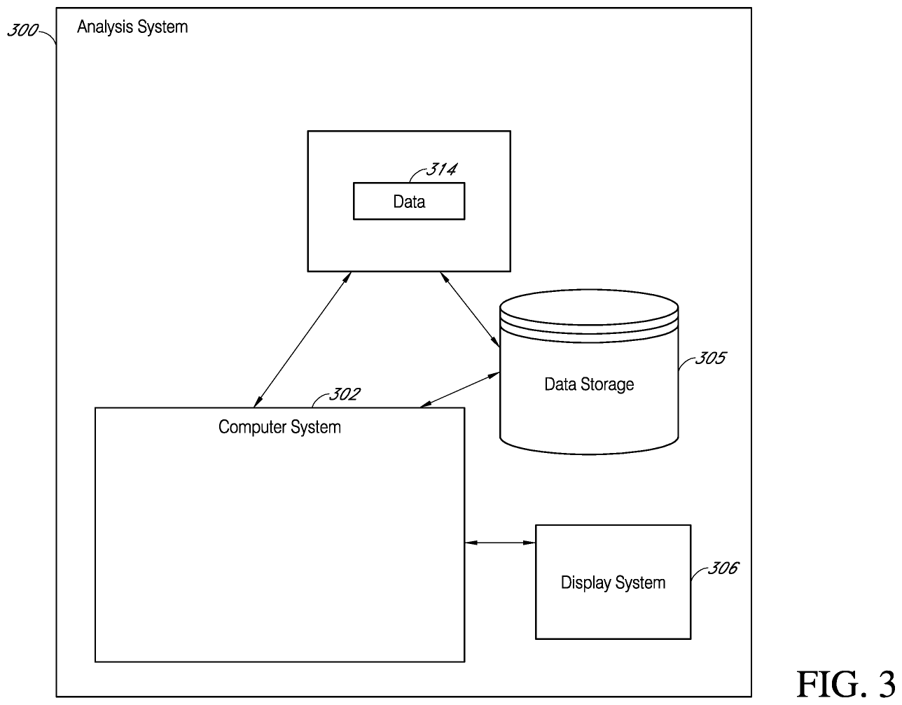

That is, as depicted in FIG. 3, the methods and systems disclosed herein can be implemented on a computer-based system 300. The system 300 may comprise a computer system such as computer system 302 (e.g., a computing device/analytics server). In various embodiments, the computer system 302 can be communicatively connected to a data storage 305 and a display system 306 via a direct connection or through a network connection (e.g., LAN, WAN, Internet, etc.). The computer system 302 can be configured to receive data described herein. It should be appreciated that the computer system 302 depicted in FIG. 3 can comprise additional engines or components as needed by the particular application or system architecture.

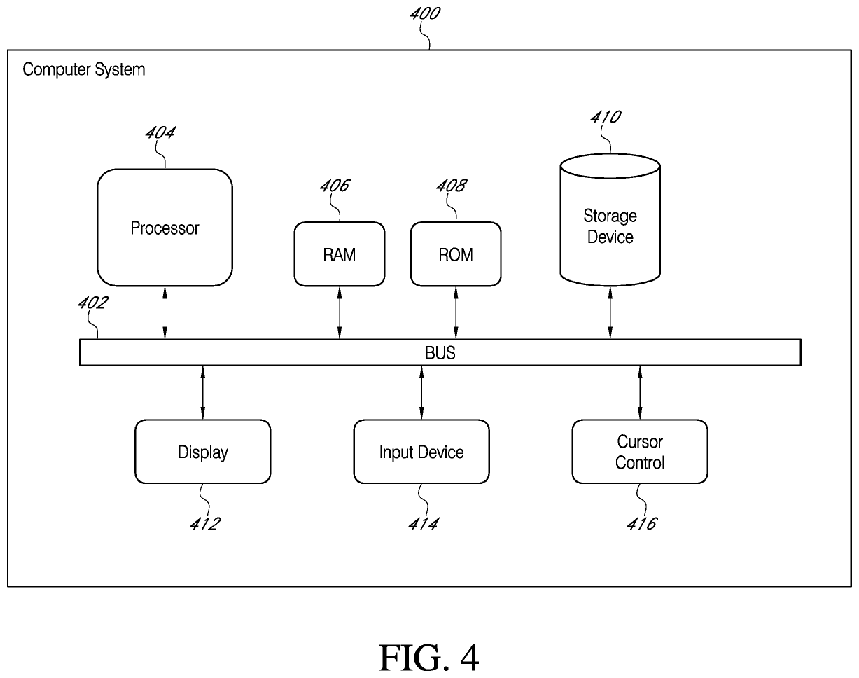

FIG. 4 is a block diagram of a computer system 400, in accordance with various embodiments of the present disclosure. Computer system 400 may be an example of one implementation for computer system 302 described herein with respect to FIG. 3. In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 412 allowing for three-dimensional (e.g., x, y and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM 408, or storage device 410 and user input provided via input device 414.

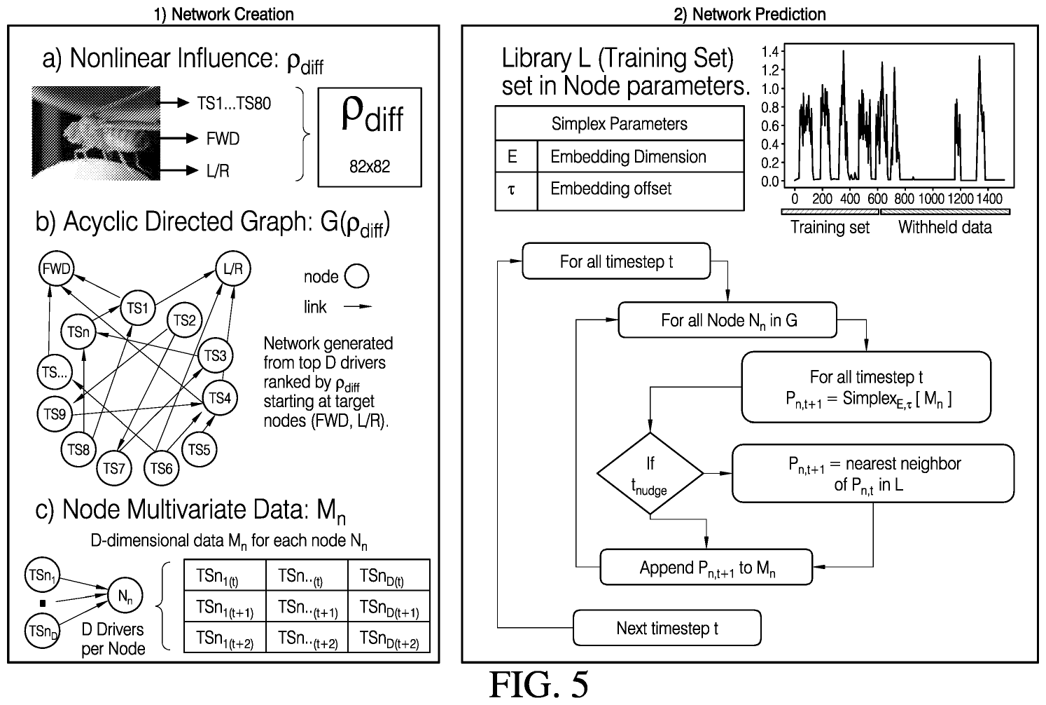

FIG. 5 is a simplified diagram of training and using a GMN. The left panel (“1) Network Creation”) shows the outputs resulting from steps 120, 130, and 140 of method 100. The right panel (“2) Network Prediction”) shows the process of step 220 of method 200.

### EXAMPLES

### Example 1: Application of GMN to Movement of Drosophila Flies

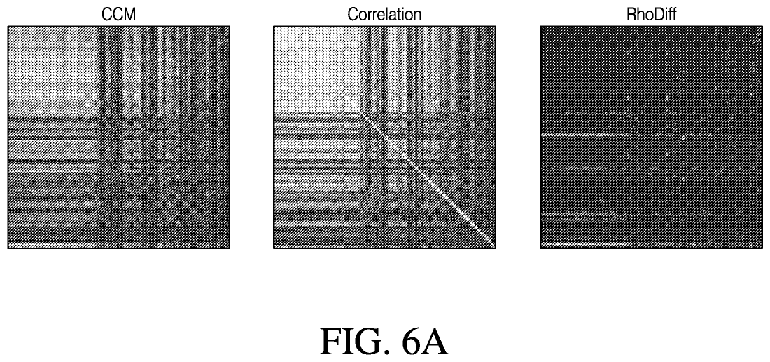

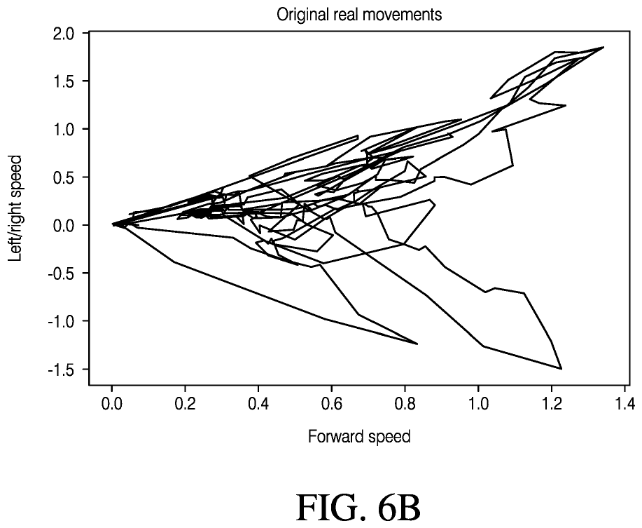

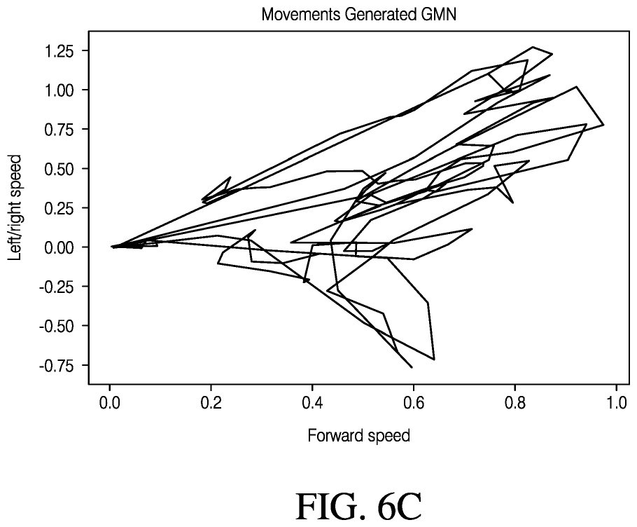

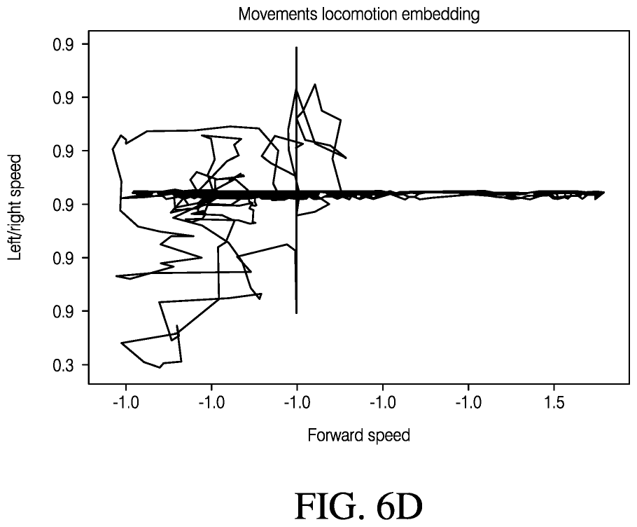

FIG. 6 shows examples of extraction of relationships between variables of a given GMN and simulation outputs of the GMN, in accordance with various embodiments of the present disclosure. FIG. 6A shows examples of CCM, correlation, and predictability (“RhoDiff”) matrices. To identify relationships between variables of a given manifold, the CCM matrix was obtained. The absolute value of the correlation matrix was subtracted to obtain the ρ_{diff} residual. This identifies the candidate time series for each embedding. FIG. 6B shows an example phase portrait of the fly forward and left-right speeds. FIG. 6C shows an example output of the GMN. As is easily seen in FIGS. 6B-C, the phase portrait is visually similar to the output. FIG. 6D shows an embedding of prior movements of the fly. As seen in FIG. 6D, this embedding fails to generate realistic fly speed changes and suggests that the information may come from the brain and not from the walking patterns themselves.

To proceed from these individual time series to a network, it may be helpful to first understand how the time series relate to one another. To this end, convergent cross mapping (CCM), a Takens theorem based causal inference method that can detect nonlinear causation between time series (even without correlation), was used. CCM was run on all pairs of time series in the training data, producing for each pair a scalar value, ρ_{ccm}_{ᵢⱼ} that quantifies the nonlinear causal effect of time series j on time series i. To extract from these causal effects the most salient, non-trivial and nonlinear, the absolute value of the linear (Pearson) correlation between each pair of (training) time series was subtracted from p_{ccm} to generate a new matrix ρ_{diff}_{ᵢⱼ}=p_{ccm}_{ᵢⱼ}−|p_{corr}_{ᵢⱼ}|. Thus ρ_{diff} quantifies the nonlinear predictability skill over and above linear correlation, thereby identifying the most important causal links between the time series. This information was then used to combine timeseries into manifolds, and to link these manifolds into a network.

The manifold network was constructed around a given target variable i (e.g. forward movement of the fly) as follows. First, the various time series j with the highest values of were identified. A number E−1 of these were selected, such that the final dimensionality of the manifold was E (each time series will contribute one dimension). The selected variables together with the target variable then defined a manifold that was eventually be used to predict the target variable i using the Simplex projection method. To move from this single manifold to a manifold network, notice that each of the variables j that contributed to the manifold used to predict variable i, can in its turn also be predicted from another manifold that includes the variables that are most causally relevant to j, by the same scheme. Thus, the manifold network was grown outwards from a target variable (e.g. forwards movement) by linking manifolds together via their prediction outputs.

In an example, the manifold, or attractor, of each node in the network was defined by the training data time series (“library”) for the variables used at that node. This time sequence of points in an E dimensional space could be used to predict forwards in time from a novel point in this same E dimensional space using the Simplex projection method. A brief example will clarify this prediction process. Suppose an attractor at a node that predicts left-right motion is defined using the training data for left-right motion, and 3 other variables. Given a new point in this 4-dimensional space (i.e. simultaneous values of left-right motion and the three other variables), the GMN tries to predict the values of those variables at the next time step. To do this, the closest 5 points on the training attractor (which define a 4-dimensional simplex) were found. Those training points were then iterated forward by one time step. The predicted next position of the novel point was then center of mass of the forward-iterated simplex. The predicted value of left-right motion was thus simply the left-right motion component of this predicted vector. When running the network in predictive mode (e.g., for model testing and validation) the novel point at each time step will come from testing set data. In generative mode, the novel point at each time step comes from the previous predicted output for each variable. To avoid instabilities when operating in generative mode, the novel point may be nudged to its nearest neighbor on the training data manifold surface every s time steps.

In an example, at the network level, two details may be addressed regarding data input and synchrony. Each node (or manifold or attractor) in the network was used to predict one target variable. This target variable was the only one fed directly into the node (either from the previous prediction in generative mode, or from a test dataset in prediction mode). The other variables corresponding to the node's attractor were fed in from its upstream nodes, which means that they needed to be fed in one timestep ahead to maintain synchrony with the downstream node. For sets of nodes connected in a loop, this synchrony requirement may not be meetable, and so it can be ignored, sending instead the predicted value at the current timestep to the downstream node. This method is described for example in Algorithm 1, below.

Algorithm 1 Generative Manifold Network algorithm

```
INPUT: set of training time series {xᵢ}
INPUT: set of target node indices tni = {i₁, i₂, ...}
INPUT: manifold embedding dimension E
INPUT: number of points to generate ng
INPUT: replacement window size rw
Calculate pairwise CCM matrix ρccm [symbol]  = CCM(x [symbol] ,x [symbol] )
Calculate pairwise Pearson correlation matrix ρcorr [symbol]  = corr(x [symbol] ,x [symbol] )
Causality matrix ρ_{diff} [symbol]  = ρccm [symbol]  - |ρcorr [symbol] |
Directed graph G
Stack nodes = tni
while nodes not empty do
  i = pop(nodes)
  Top E - 1 driving nodes tdd = columns in x [symbol]  corresponding to top E - 1 values in CM [symbol]
  for all drivingNode ϵ tdd de
    if edge driving Node → i does not introduce cycle in G then
      add primary edge drivingNode → i to G
    else
      add secondary edge drivingNode → i to G
    end if
    add drivingNode to nodes
  end for
end while
for all node n ϵ G do
  Let pd = all nodes u such that u → n and u → n is marked a primary edge
  Let sd = all nodes u such that u → n and u → n is marked a secondary edge
  Let library = {x [symbol] } for i in row indices corresponding to node & and nodes in pd ∪ ad.
  for all d ϵ pd do
    Shift all columns in library row d one forward, such that library[d,t] becomes
    library[d, t [symbol] ]
  end for
  Build Simplex model Simplex [symbol]  on training data library
end for
Generated points matrix gp = |G| x ng
Calculate topological ordering of G to
for [symbol] =0; [symbol]  < ng; [symbol] ++ do
  for all node i ϵ to do
    With [symbol] 0's pd. sd as defined above, collect pd [symbol] ,sd [symbol] [symbol]    from gp
    Calculate ng(i, t + 1] = Simplex [symbol] (pd [symbol] sd [symbol] [symbol] )
  end for
end for
for [symbol] =0 [symbol]  ng: [symbol]  += rw do
  Replace gp [symbol]  with its nearest rw length point in traning, using only rows tni for the
distance
  calculation
end for
return gp [symbol]
indicates data missing or illegible when filed
```

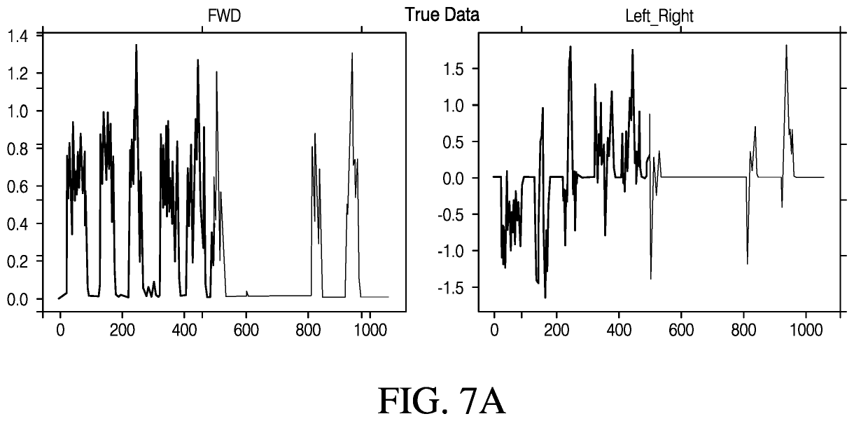

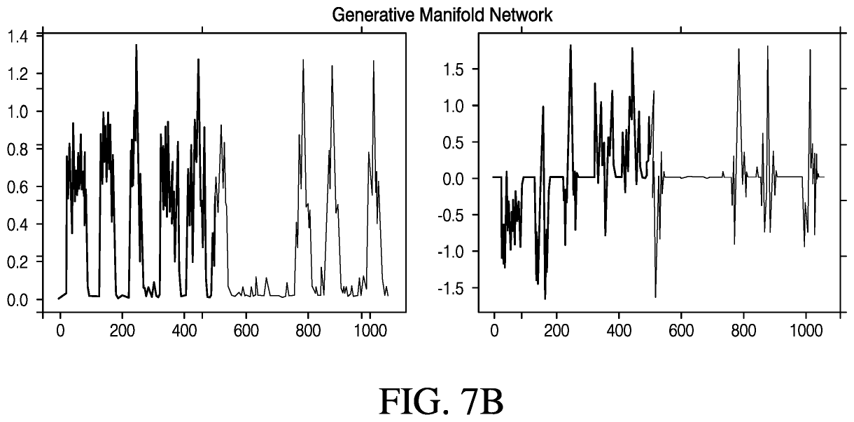

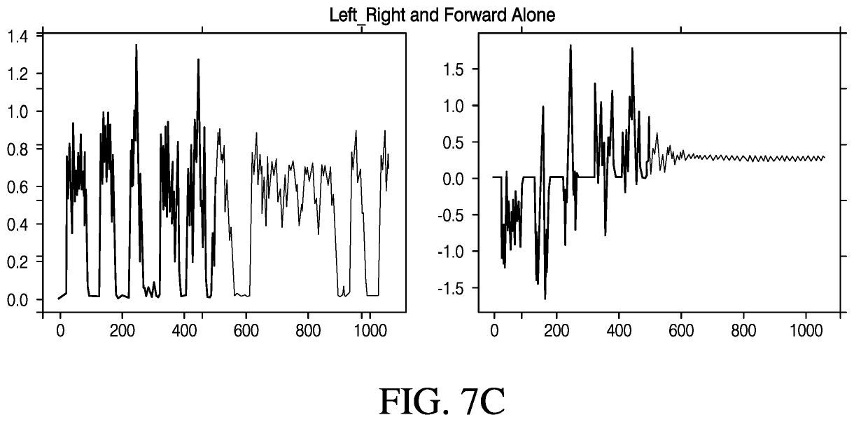

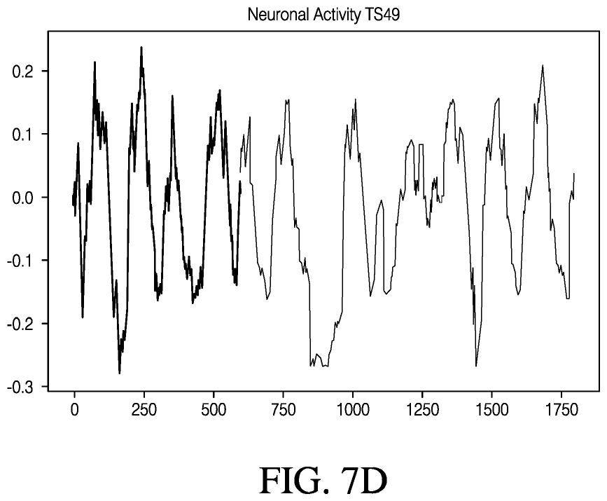

Continuing with Example 1, FIG. 7 shows illustrative inputs and outputs of an example GMN, in accordance with various embodiments of the present disclosure. FIG. 7A shows an example of real forward and left right speeds of Drosophila locomotion. FIG. 7B shows an example of GMN generated speed time series. Note the emergence of pauses that are not in the training interval. FIG. 7C shows an example of Simplex generated time series from an embedding of the fly movements alone. FIG. 7D shows an example of realistic neuronal activity generated by the GMN.

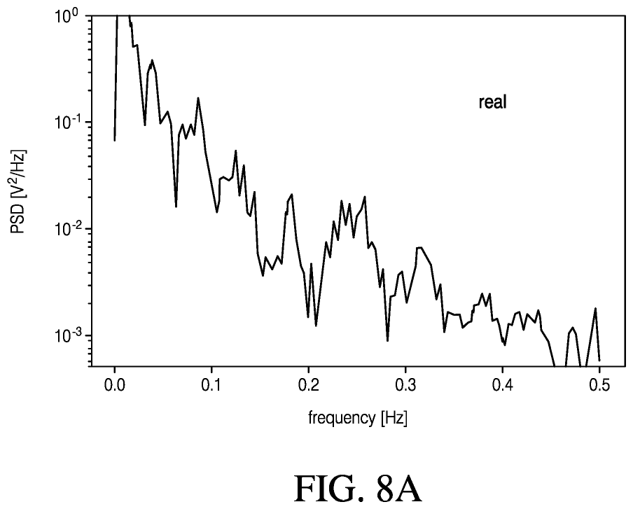

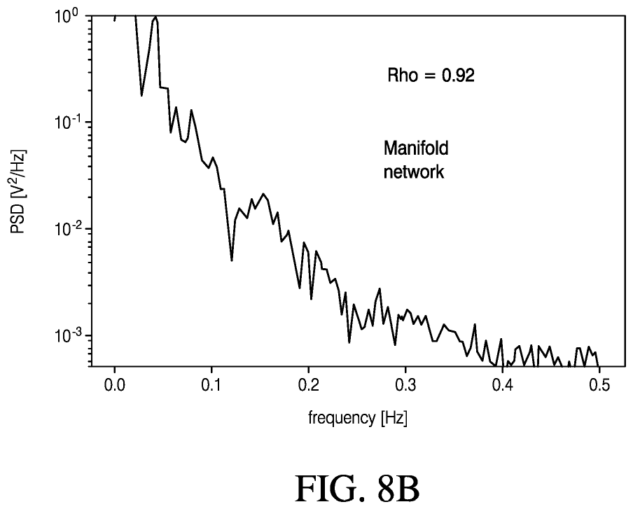

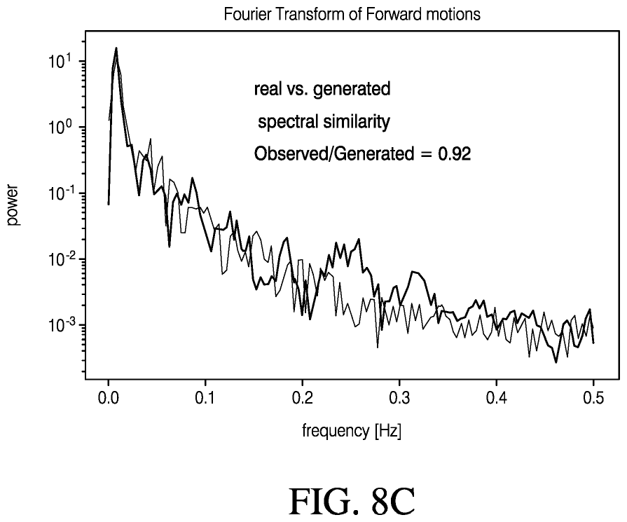

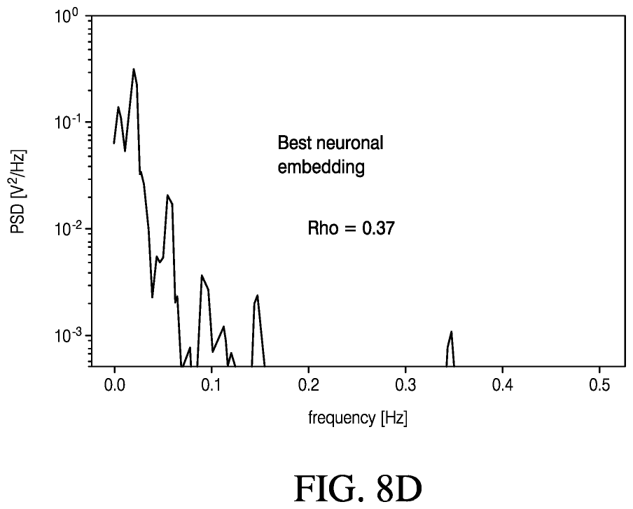

Although Takens type time delay embeddings frequently have high predictive skill, they may fail over time if predictions are used recursively to drive the network forward (FIG. 7C). Over time, predictions decrease in amplitude and eventually may converge to a constant, or to a very low-amplitude oscillation. This may be largely due to the fact that simplex projection is an averaging approach where projecting into a center of a simplex averages the result until there will be a projected point for which the nearest neighbors will never change. This is true for predicting itself as well as cross-predicting to another variable as seen in FIG. 7C, right panel, where the generated output converges to a constant. On the other hand, time series outputs of the generative manifold network (FIGS. 7B and 7D) tended to not fall either into cycles nor converge to a constant, and look superficially similar to the oscillations observed in the training set. The similarity of generated time series was not limited to movement regressors: also neural activity time series looked quite similar (FIG. 7D). To have a more quantitative measure of the properties of the forward oscillations generated compared to the real forward speed time series, the Fourier transform of the real time series was calculated and compared to the spectrum of the manifold network generated time series as well as the time series generated by the highest performing single brain area (FIG. 8). Overall the manifold network-generated time series data matched well where the cross correlation coefficient between the real spectrum and the manifold network generated spectrum was 0.92 whereas a time series from the brain area that best predicted forward movement was only able to obtain a correlation coefficient of 0.37.

Continuing with Example 1, FIG. 8 shows a comparison between real and generated movements, in accordance with various embodiments of the present disclosure. Depicted is a comparison of the Fourier transform of the fly movement comparing real, generated, and the best single neuronal predictor of forward walking speed. As seen in FIG. 8, the real and generated Fourier transforms appear to be similar. This is in contrast with the neuronal embedding, which is highly dissimilar from the real data.

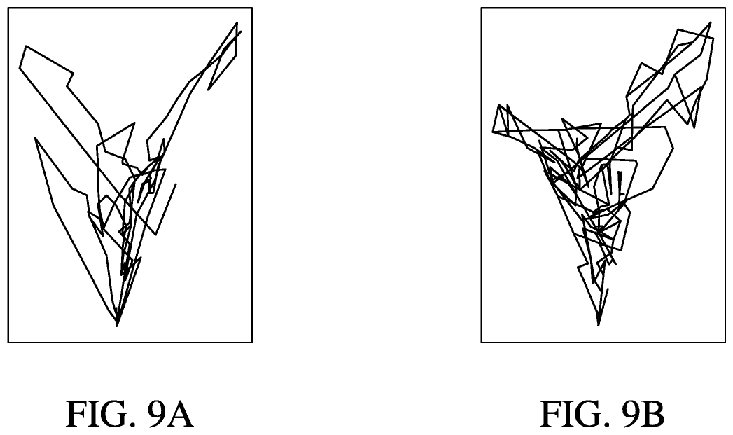

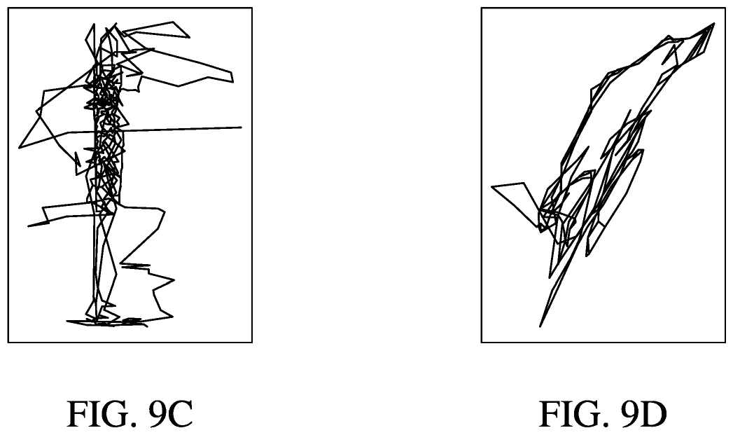

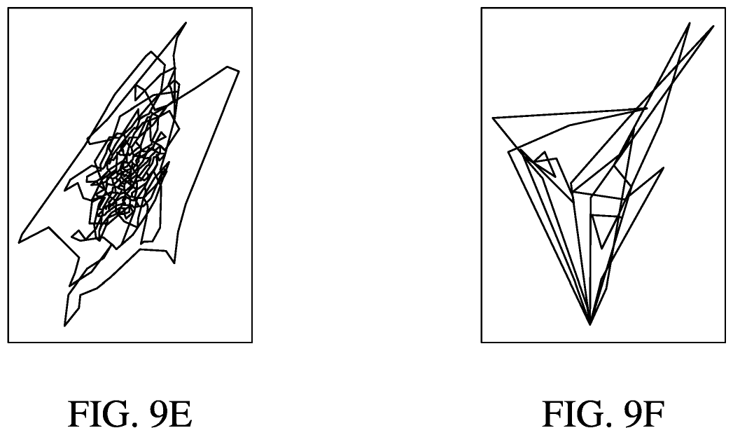

Continuing with Example 1, FIG. 9 shows a comparison of real fly movements with movements generated by different modeling approaches, in accordance with various embodiments of the present disclosure. Depicted is a comparison of the real fly speed phase portraits generated by: the GMN, an embedding of the fly left-right and forward speed, the flattened network of 82 time series, a recursive prediction vector autoregressive model, and a recursive kNearest Neighbor predictor.

The similarity of the forward and left-right phase plots of the observed fly movement was compared with the movements generated from the generative manifold network embedding. As seen in FIG. 9, both the natural and artificial phase plots have a similar overall geometry and appear to be similar. To exclude the possibility that just the walking motion of the fly itself contains sufficient information to generate realistic fly movements, the left right and forward time series alone were embedded into a predictive manifold that ran in generative mode. This phase plot failed to give the same realistic geometry, suggesting that the information for realistic movement may be contained in the neural activity patterns and not in the movements themselves.

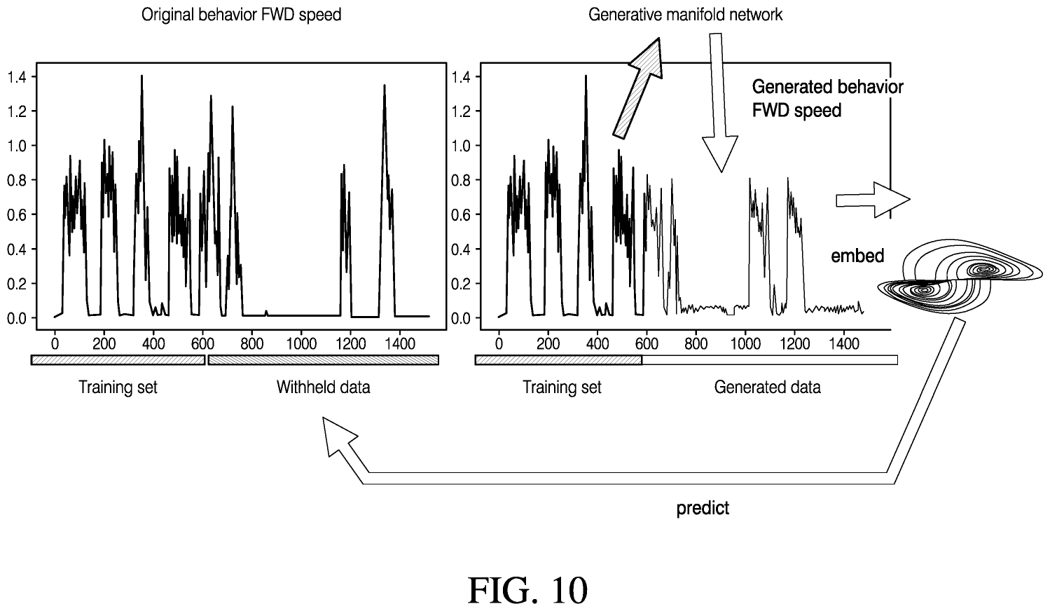

Continuing with Example 1, FIG. 10 shows a validation scheme of the GMN generated data, in accordance with various embodiments of the present disclosure. The realism of the generated data was evaluated as its prediction skill to predict out of sample withheld time series.

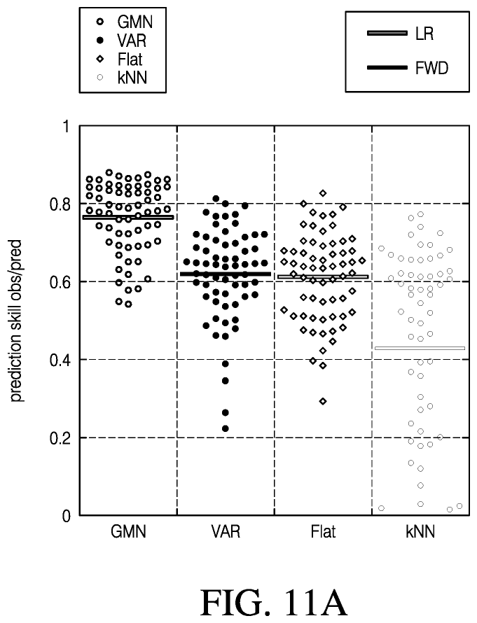

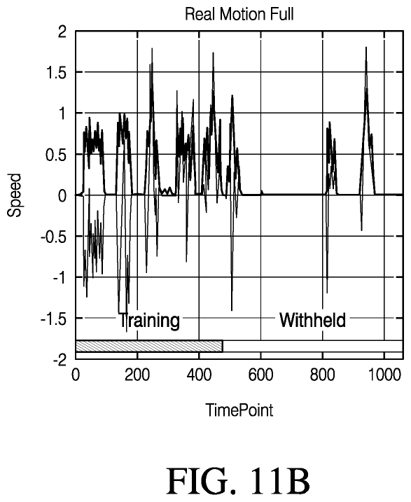

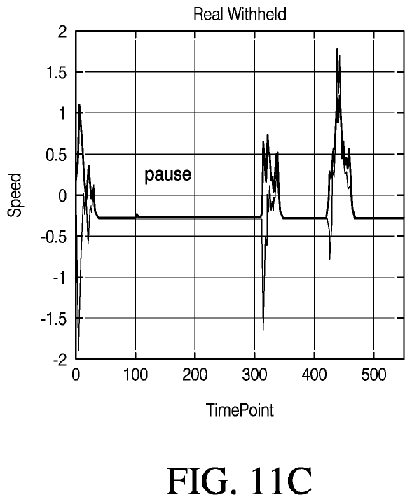

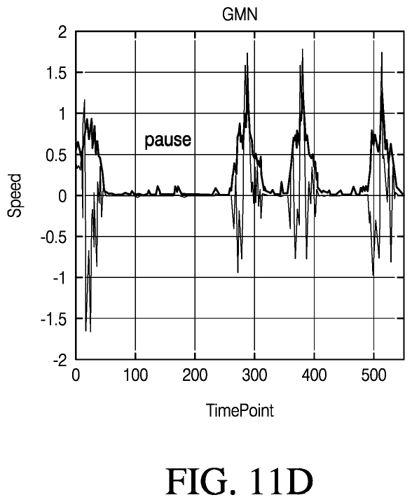

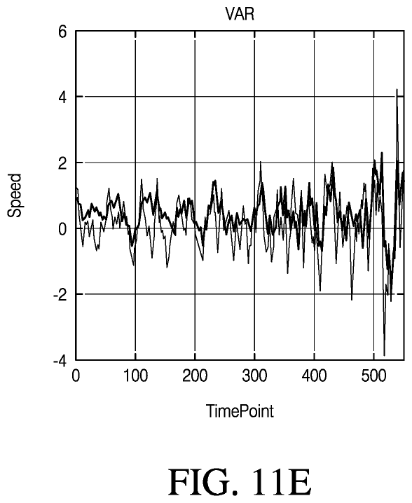

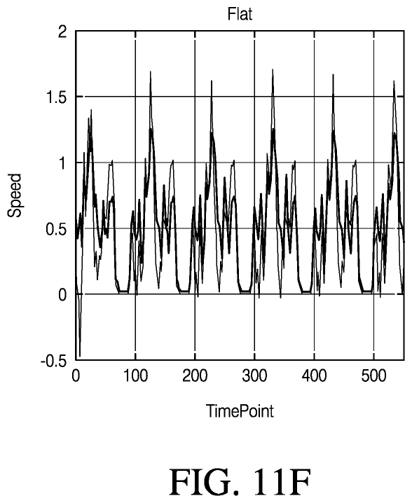

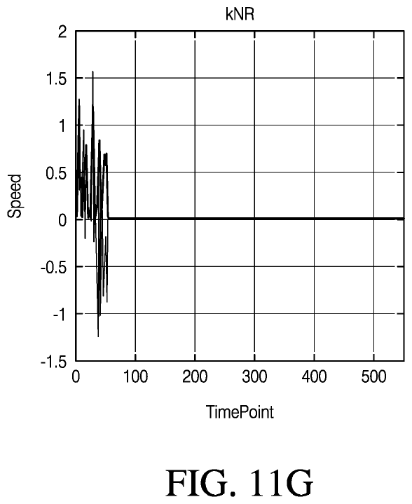

Continuing with Example 1, FIG. 11 shows the prediction skill of the proposed method compared to three other methods, to validate similarity to real motion data, in accordance with various embodiments of the present disclosure. FIG. 11A shows prediction skill of the 4 generative models on withheld data to assess realism of generated time series. FIGS. 11B-11F show time series of 4 models used in this study. In the GMN (FIG. 11D), observe the similarity in the pauses not present in the training set but present in the withheld real time series.

To build the generative manifold network model, the first 500 points of the original downsampled time series out of 1200 total time points were used as seen in FIG. 11B, left. The original time series and the network generated forward speed time series are shown. Interestingly although training set did not have long movement pause periods, the generated forward time series had a couple long low forward velocity down states. These low-speed periods were also observed in the withheld real data that was not part of the training set. This suggests that the network may contain the capability to generate behaviors that are not strictly present in the training set and that it is not simply reproducing behaviors in the training set. FIG. 6D shows the generated time series for forward speed and left-right speed for a stretch of generated data. As one can observe, these rest periods actually experience some unnatural low-amplitude oscillations. These oscillations might be masked in the real flies and might occur as subthreshold events in the real fly. Since none of these features appear in the original training set period, these observations suggest that this type of network may be capable of generating emergent properties rather than just reproducing features of the training sets through features hidden in the network embeddings.

In order to investigate the possible properties of the network, the generative manifold network output was compared to the real withheld data, the output of an embedding of the left-right and forward speeds, and a flat network which is an embedding of all 80 time series of the entire dataset into a 80-dimensional embedding. Shown are the forward and left predictions for all 4 models. As can be seen both the left-right and forward embeddings, generated time series fall into cyclical behaviors from which they seem to be unable to get out, or converge to a constant value. This did not appear to happen to the generative manifold network. Comparison of individual the left right and forward generated movements show that the generative manifold network appears to be the most realistic with the embeddings of the movements alone based on its past history being the most dissimilar. The fact that movements alone cannot generate realistic movements in a multidimensional embedding suggests that motions may not encode their own future movements and it is not Markov-chain-like. In addition, the fact that the generative manifold network performs better than the flat network (which contains, in principle, all the information within the network) suggests that the information gating within the network may play an important role.

One way to test that the time series that are generated by this network are realistic is to show that the generated time series have the same fundamental dynamics as the observed data and thus can be used as training data for the prediction of withheld real data: successful predictions indicate shared dynamics. For this test, time series data were generated for every brain area as well as simultaneous time series of the forward and left right movements and the generative manifold network was to: 1) an autoregressive linear model, 2) the flat network and 3) an 80-dimensional nearest neighbor predictor. Results in FIG. 6 show that the generative manifold network outperforms all three: the flat network, the autoregressive linear model as well as the K-nearest neighbor model by significant margins indicating that GMN produces synthesized time series that have dynamical features that may be closer to those in the observed data than the alternatives. FIG. 9 shows a sample of the predictions from the GMN, the embedded left-right and forward motion speeds of the fly and the flattened network which embeds all time series into a single manifold. The ability of the generated data to skillfully predict withheld real data from the same brain areas shows that the generated data is indeed quite similar to data directly obtained from the fly (FIGS. 9, 11).

Presented herein are methods based on the generalized Takens theorem for uncovering such a mapping. This approach uses causal inference to reduce dimensionality and select the variables that are most informative and least redundant to identify candidate variables that can be used to build a network of low-dimensional manifolds. The networks connect through shared nonredundant information which will have a topology that is very different from the network topology of the physical connectome (which is expected to be highly correlated in time). To use an analogy with a city, this is not a street map of the city but the relationships within the city parts extracted from the traffic patterns. Thus, the causal network generated will capture relationships in the brain that reflect information flow but that may not have the same network structure as the physical brain. The nonredundancy aspect of this approach may be important in order to properly “unfold” the manifolds so points will be optimally separated to avoid singularities and ambiguity in the predictions. In some embodiments, dropping nodes within the network may allow identification of brain areas that are important for a given mapped behavior or activity. In the current work, the ρ_{diff} matrix was sufficiently sparse that redundancy was naturally avoided in the manifold generation scheme. In some embodiments, an additional step may be employed to avoid redundancy in the manifold variable selection, such as clustering the variables according to their causal relationships, and enforcing the constraint that each variable in each manifold come from a different cluster.

Previous approaches using low-dimensional manifolds to generate simulated behaviors have generated, for example, realistic human locomotion, but based on kinematic data of motion capture using the position of joints, which is mechanistically closer to the generation of movements. The left-right and forward speeds herein by contrast are the aggregate output of the complex motion of six legs and multiple joints. A low-dimensional embedding approach was also able to map brain activity to animal position in an arena. With reservoir computing and low-dimensional embeddings it has been observed that when driven recursively, these models frequently fall into short, repeating, possibly infinite cycles.

With generative manifold networks, locally “trapped” dynamics and manifold prediction collapses seem to be largely avoided, especially in well-connected nodes. The remarkable pauses that the networks generated that were not in the training data (but that were in the testing data) were not at a nonzero value close to the median of the distribution, and they were of limited duration, after which the network reinitiated normal-looking, large-amplitude oscillatory behaviors. It is possible that these could be an emergent property of hidden information contained within the network that was not explicitly present in the training set. The dataset used to generate this model was essentially from a fly to which no stimulus was given and allowed to freely move that should be akin to a “default network”. Some embodiments may incorporate artificial sensory input and use datasets with a directed behavior such as presentation of images or odors to drive behavior. Going forward, the model embodiment discussed herein can be scaled up to whole-brain data sets at single-neuron resolution to obtain a generative whole brain simulation. As this approach is scale-independent, one can also use this approach with lower-resolution data to assess the informational content of a dataset as judged by its capability to generate the observed behavioral task in settings like EEG or fMRI where both temporal and spatial resolution are limited. In addition to neuroscience, this type of network architecture can be generic and applicable to any nonlinear network, so it may have wide ranging applications in other areas.

The GMN technology is capable of reproducing any arbitrary behavior from recorded neural data provided it is sufficiently complete and contains information on the key variables relevant to the task.

Insofar as this technology advances closer to the possibility of downloading a brain into a computer (a naturally seeded AI that can then generate natural behaviors), the consequences could be far reaching. Our work could also impact brain computer interfaces as it should perform much better than the currently prevalent linear decoder scheme in the public domain of brain computer interfaces.

A data-driven nonlinear mapping with predictive capability is applicable across domains. Certainly in state estimation and prediction applications, which, are everywhere: autonomous control (auto-pilot, neural prosthesis, drones, error-correction, etc. . . . ); and in modeling, data-discovery, and dimensional reduction.

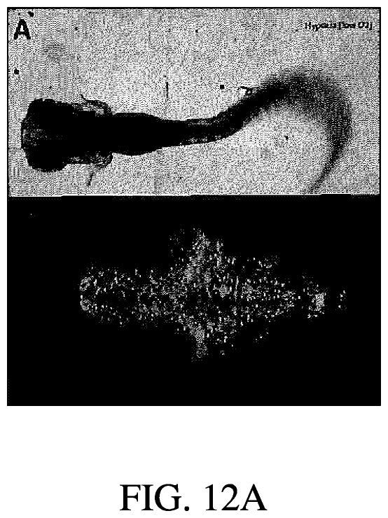

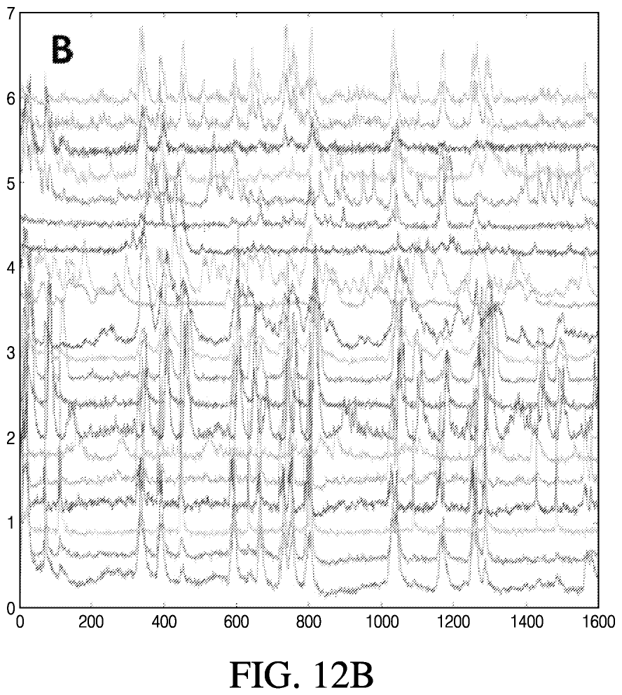

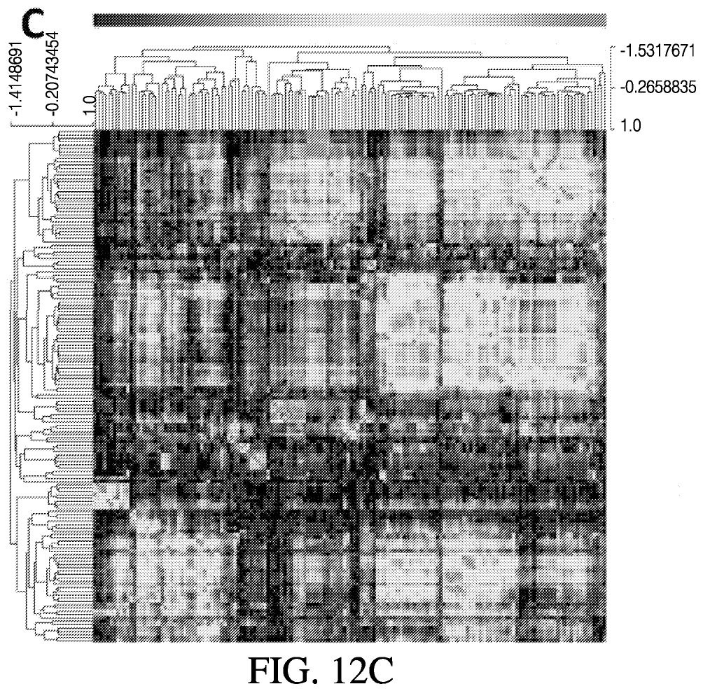

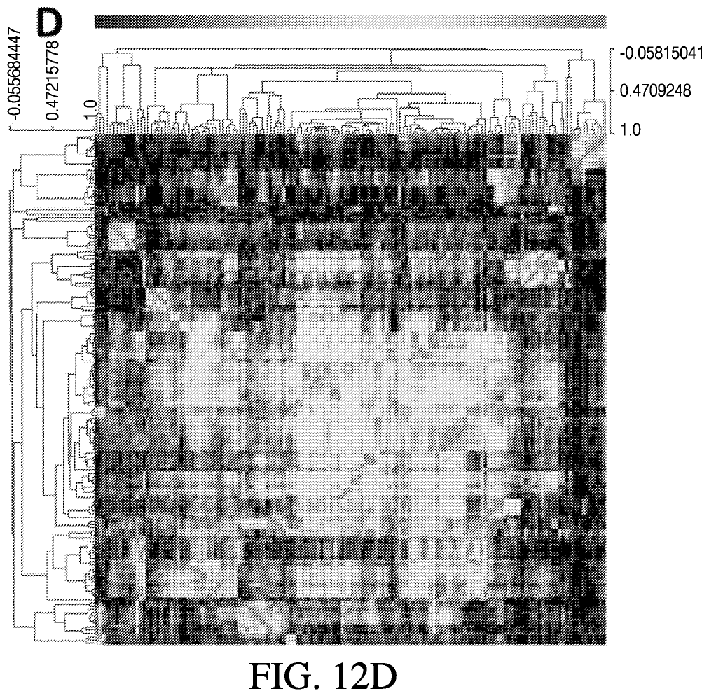

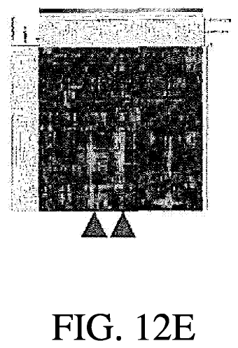

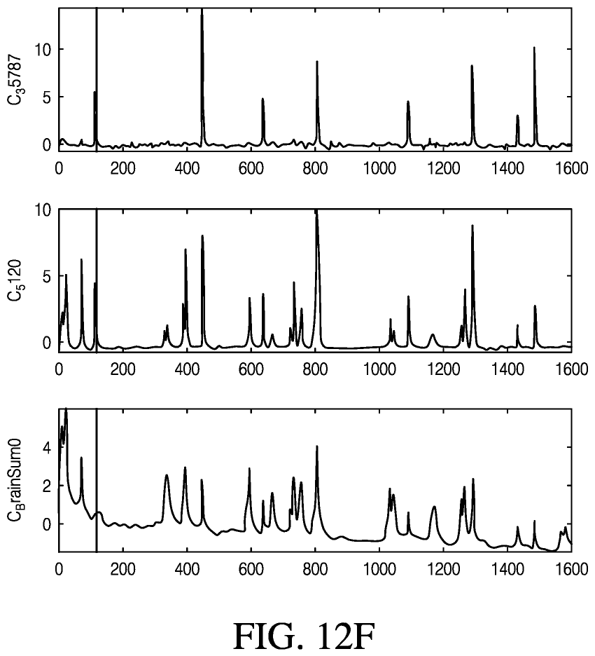

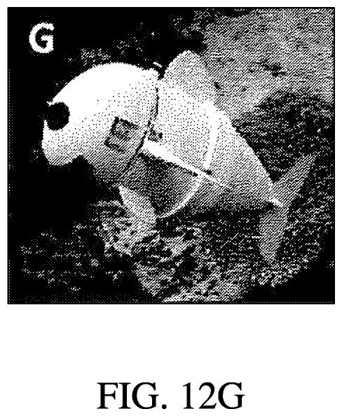

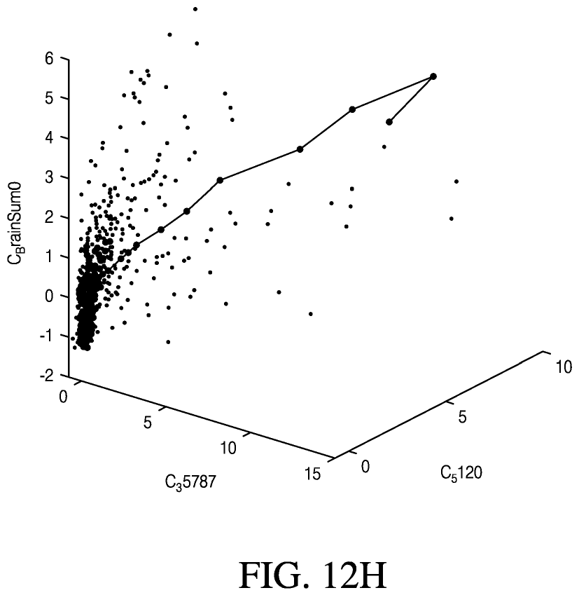

Continuing with Example 1, FIG. 12 shows an example of generating a model of neural activity and behavior of an organism for download into a robot, in accordance with various embodiments of the present disclosure. FIG. 12A shows examples of turning behavior and whole-brain neural activity of SPIM GCAMP6 imaging. FIG. 12B shows examples of representative time series extracted from microscopy images. FIG. 12C shows examples of CCM inferred causal relationships between 154 representative neurons during “default” normoxia conditions. FIG. 12D shows examples of causal relationships during the hypoxia escape response are much more simplified and coherent. FIG. 12E shows examples of vertical columns that identify presumed sensorimotor integrators. FIG. 12F shows examples of GCAMP6 time series of two integrators and motor activity. FIG. 12G shows an example of a robotic fish: SOFI soft robotic fish. FIG. 12H shows an example of an identified manifold predicting fish turn behaviors which have at least three attractor states.

It was typically considered impossible, due to the limited understanding of a brain, to use the activity of a brain to control a robot. There is a need for an approach that does not require an a priori understanding of the computational complexity of the brain but can achieve this by extracting information from empirical observations. In a non-limiting example, using recent developments in live microscopy, computational causal inference as well as AI, two alternative methods were employed to “download” the brain activity of a zebrafish larva, imaged by light sheet microscopy at single cell resolution, into a deep neural network to control a fish robot.

The brain activity relationships were extracted using the Sugihara chaos-theory-based methods run on the Japanese ABCI supercomputer. Unique to this approach is that it allows the extracted relationships to be transferred directly onto artificial neural networks.

The idea of capturing an organism's brain activity and reproducing its activity in a computer to guide the actions of a robot has been a longstanding idea but thought to be presently technically not achievable due to a large number of technical hurdles as well as gaps in our understanding of basic neuroscience. In its most simple form, a typically imagined approach involves the determination of the complete connectome, (i.e. the physical connectivity of the nervous system) in which the synaptic strengths can also be determined and then initialize the complete network with a snapshot neural activity pattern that will propagate and maintain all normal brain activity. In the case of the human brain the complexity imagined is that of 10\hat{ }11 neurons with 10\hat{ }15 synapses, for which we would have to get a connectivity map, synaptic strength and initial state all simultaneously, with little error, in order to ensure function. This is generally thought to be a problem of at least 10\hat{ }11 dimensions (one for each neuron of the human brain), which may be an intractably large number.

Even in the case of the larval zebrafish with ˜120,000 neurons, this 120,000-dimensional problem is presently not solvable in this form, as we do not have the connectome of the larval zebrafish, nor do we have the synaptic strengths, which are pieces of information required to reconstruct a whole brain computationally in this manner Complicating this notion, recent work from the mouse brain shows that 97% of possible physical connections exist within the network of analyzed areas of the mouse cortex. This suggests that the mouse brain is much like an untrained computational artificial neural network which starts with all possible connections realized. Only with training are these then pruned and weights assigned to become functional. This suggests that activity rather than physical connectivity is key to understand function. Using an analogy of a city; to understand how a city works it is the traffic pattern that is more important than the street map. Thus, a connectome-alone-based approach to computationally reconstruct a brain is unlikely to create a good representation of a functional brain in silico.

The present disclosure provides an approach that does not require an a priori understanding of the computational complexity of the brain but can extract information from empirical observations. In a non-limiting example, this approach uses neural activity imaging data of an entire brain at single cell resolution in a behaving larval zebrafish (a transparent vertebrate) to extract all relationships in an intact vertebrate brain. To achieve this, whole-brain neural activity patterns in multiple animals experiencing hypoxia were recorded using a Selective Plane Illumination Microscope (SPIM). Data was obtained from the entire 5-day-old larval brain (120,000 neurons) at 2 Hz in response to hypoxia (an example frame is shown in FIG. 12). The brain activity relationships were extracted using unique novel computational methods of causal inference and interaction strength quantification on the world's 3rd fastest AI super computing platform (5th by LINP-ACK TOPS00 standard) AI Bridging Cloud Infrastructure (ABCI) in Japan. The Sugihara lab at Scripps Institution of Oceanography, UC San Diego has developed a suite of mathematical tools based on the generalized Takens embedding theorem collectively named Empirical Dynamic Modeling (EDM) for the analysis of nonlinear time series. Among these, Convergent Cross Mapping (CCM) allows the inference of causation from nonlinear time series even with substantial noise and complete absence of correlation. The present disclosure employs CCM and other tools from the empirical dynamical modeling framework for the inference of existence, strength and sign of causal relationships within the neural activity network of the transparent larval fish brain. CCM determines whether and how much causality exists between individual neurons. The adjacency in the network was determined by time delay cross-mapping as well an epistatic analysis like procedure. As a test case, multiple data sets of lengths around 1600-time steps were collected at 2 Hertz which contain 50,000-100,000 active neurons in each case. The generated time series were suitable for network inference using the EDM framework and thus demonstrated a proof of principle feasibility using a 154-neuron subset. (see FIG. 12). CCM calculations may be computationally expensive, and performing these on a latest-generation MacBook Pro laptop, for a single 70,000 neuron dataset, could require over 2000 years to complete. In order to complete this task, the study was performed on the Japanese ABCI supercomputer. The inferred causal network of neural activity was then used to build the generative manifold network. The approach to download an animal's brain from its activity, was based on a principle in which a dimensionality reduction approach can produce manifolds on which surface one can decode neural activity into positions in an arena for a free moving rat from about 100 single unit recordings. As a proof of principle in a similar way, turns in response to hypoxia from the activity of two sensorimotor integrators were used to decode and predict turning activity of a fish larva exposed to low oxygen (see FIG. 12). The decoded map showed a low-dimensional manifold with at least 3 attractor states that allow decoding of the fish's turning behavior. Coupling this map to those which are the inputs of the integrating neurons allowed the control of a fish robot based on rules defined by the fish sensorimotor integration neural activity.

A cyber-physical complete model of an organism for a particular behavior, that can capture all relevant task-specific relationships present in the brain, may significantly help the bridging of the brain-mind gap. An obvious advantage of a cyber-physical model, in the form of a robot, is that it allows in principle for the manipulation of every neuron. This may enable the computational testing of necessity and sufficiency (i.e. the minimal network required for a task/function) in neuroscience which up to now has not been possible. Up to now, lesions or inactivation of brain areas have tested for necessity and electrical stimulation or its optogenetic equivalent, test for induction ability. No experimental approach thus far allows for the investigation of necessity and sufficiency (minimal networks) at a whole-brain scale in a vertebrate. This approach may allow a holistic interrogation that will help in bridging the brain mind gap.

The idea to download a brain to a computer and replay it in silica is an old one. Despite the concept having been long standing, it has not been realized because of a lack of technical means. This disclosure provides a technically feasible and plausible path to achieve this goal. Longer term payoffs are numerous: e.g. if additional behaviors are integrated, a more general type of intelligence may be achievable with this general framework. Furthermore, both approaches provide the basis of novel neuromorphic architectures for the development of self-learning algorithms and the development of novel forms of biologically inspired AI that should have many uses.

Accordingly, it can be seen that the GMNs described herein fill a long-standing need in the art, by permitting more accurate simulation and modeling of complex systems such as brains. The logical operations making up the embodiments of the technology described herein are referred to variously as operations, steps, objects, elements, components, or modules. Furthermore, it should be understood that these may occur or be arranged or performed in any order, unless explicitly claimed otherwise or a specific order is inherently necessitated by the claim language.

It should further be understood that the described technology may be employed in diverse applications including but not limited to the prediction and simulation of brain activity, internet traffic, highway traffic, interconnected citywide activities, weather, stock market, commodities markets, hedge funds, investment banks, online behavior, etc. In addition, the system can be used to download representations of a real or simulated brain into a real or simulated robot, or a simulated body.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the generative manifold network system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary embodiments of the generative manifold network system as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

### RECITATION OF EMBODIMENTS

Embodiment 1. A method comprising:
a) receiving a plurality of neural time series signals, each neural time series signal associated with at least one neural signal resulting from at least one location in a brain of a subject;
b) receiving at least one behavioral time series signal, the behavioral time series signal associated with at least one behavior of the subject;
c) determining a plurality of causality measures between each pair of neural time series signals and between each neural time series signal and the at least one behavioral time series signal; and
d) determining, from the plurality of causality measures, a subset of the plurality of neural time series signals that correlate with the at least one behavior.

Embodiment 2. The method of Embodiment 1, further comprising:
e) receiving at least one neural signal associated with a time point; and
f) determining, based on the subset, at least one simulated behavior associated with the at least one neural signal.

Embodiment 3. The method of Embodiment 1 or 2, wherein (c) comprises: (i) determining a mapping matrix between the plurality of neural time series signals and the at least one behavioral time signal; (ii) determining a correlation matrix between each pair of neural time series signals and between each neural time series signal and the at least one behavioral time signal; and (iii) subtracting the correlation matrix from the mapping matrix to thereby form a predictability matrix.

Embodiment 4. The method of Embodiment 3, wherein the mapping matrix comprises a convergent cross mapping (CCM) matrix.

Embodiment 5. The method of Embodiment 3 or 4, wherein the correlation matrix comprises a linear correlation matrix.

Embodiment 6. The method of any one of Embodiments 3-5, wherein the correlation matrix comprises a Pearson correlation matrix.

Embodiment 7. The method of any one of Embodiments 3-6, wherein (d) comprises: (i) determining an acyclic directed graph based on the predictability matrix; and (ii) determining, from the acyclic directed graph, the subset.

Embodiment 8. The method of any one of Embodiments 2-7, wherein (f) comprises: (i) determining, based on the subset, a plurality of simulated neural signals associated with a plurality of simulated time points, each simulated time point occurring at a later time than the time point; (ii) determining a geometric weighting between the plurality of simulated neural signals; and (iii) determining, based on the geometric weighting, the at least one simulated behavior.

Embodiment 9. The method of Embodiment 8, wherein the geometric weighting comprises a simplex weighting.

Embodiment 10. The method of Embodiment 8 or 9, further comprising repeating (i)-(iii) to determine a plurality of simulated behaviors.

Embodiment 11. The method of any one of Embodiments 1-10, further comprising measuring the plurality of neural time series signals and the at least one behavioral time series signal.

Embodiment 12. The method of any one of Embodiments 2-11, further comprising measuring the at least one neural signal associated with the time point.

Embodiment 13. The method of any one of Embodiments 1-12, wherein each neural time series signal is selected from the group consisting of: an evoked potential signal, a far-field evoked potential signal, a near-field evoked potential signal, a single-neuron extracellular signal, a multi-neuron extracellular signal, a microelectrode signal, a microelectrode array signal, a sharp electrode signal, a patch-clamp electrode signal, an optical signal, a fluorescence signal, an intrinsic optical change signal, an electroencephalography (EEG) signal, a magnetoencephalography (MEG) signal, a magnetic resonance imaging (MRI) signal, and a functional MRI (fMRI) signal.

Embodiment 14. A system comprising:

a non-transitory memory; and

one or more processors coupled to the non-transitory memory and configured to read instructions from the non-transitory memory to cause the system to perform operations comprising:
a) receiving a plurality of neural time series signals, each neural time series signal associated with at least one neural signal resulting from at least one location in a brain of a subject;
b) receiving at least one behavioral time series signal, the behavioral time series signal associated with at least one behavior of the subject;
c) determining a plurality of causality measures between each pair of neural time series signals and between each neural time series signal and the at least one behavioral time series signal; and
d) determining, from the plurality of causality measures, a subset of the plurality of neural time series signals that correlate with the at least one behavior.

Embodiment 15. The system of Embodiment 14, wherein the operations further comprise:
e) receiving at least one neural signal associated with a time point; and
f) determining, based on the subset, at least one simulated behavior associated with the at least one neural signal.

Embodiment 16. The system of Embodiment 14 or 15, wherein (c) comprises: (i) determining a mapping matrix between the plurality of neural time series signals and the at least one behavioral time signal; (ii) determining a correlation matrix between each pair of neural time series signals and between each neural time series signal and the at least one behavioral time signal; and (iii) subtracting the correlation matrix from the mapping matrix to thereby form a predictability matrix.

Embodiment 17. The system of Embodiment 16, wherein the mapping matrix comprises a convergent cross mapping (CCM) matrix.

Embodiment 18. The system of Embodiment 16 or 17, wherein the correlation matrix comprises a linear correlation matrix.

Embodiment 19. The system of any one of Embodiments 16-18, wherein the correlation matrix comprises a Pearson correlation matrix.

Embodiment 20. The system of any one of Embodiments 16-19, wherein (d) comprises: (i) determining an acyclic directed graph based on the predictability matrix; and (ii) determining, from the acyclic directed graph, the subset.

Embodiment 21. The system of any one of Embodiments 15-20, wherein (f) comprises: (i) determining, based on the subset, a plurality of simulated neural signals associated with a plurality of simulated time points, each simulated time point occurring at a later time than the time point; (ii) determining a geometric weighting between the plurality of simulated neural signals; and (iii) determining, based on the geometric weighting, the at least one simulated behavior.

Embodiment 22. The system of Embodiment 21, wherein the geometric weighting comprises a simplex weighting.

Embodiment 23. The system of Embodiment 21 or 22, wherein the operations further comprise repeating (i)-(iii) to determine a plurality of simulated behaviors.

Embodiment 24. The system of any one of Embodiments 13-23, wherein each neural time series signal is selected from the group consisting of: an evoked potential signal, a far-field evoked potential signal, a near-field evoked potential signal, a single-neuron extracellular signal, a multi-neuron extracellular signal, a microelectrode signal, a microelectrode array signal, a sharp electrode signal, a patch-clamp electrode signal, an optical signal, a fluorescence signal, an intrinsic optical change signal, an electroencephalography (EEG) signal, a magnetoencephalography (MEG) signal, a magnetic resonance imaging (MRI) signal, and a functional MRI (fMRI) signal.

1. A method comprising:
a) receiving a plurality of neural time series signals, each neural time series signal associated with at least one neural signal resulting from at least one location in a brain of a subject;
b) receiving at least one behavioral time series signal, the behavioral time series signal associated with at least one behavior of the subject;
c) determining a plurality of causality measures between each pair of neural time series signals and between each neural time series signal and the at least one behavioral time series signal; and
d) determining, from the plurality of causality measures, a subset of the plurality of neural time series signals that correlate with the at least one behavior.

2. The method of claim 1, further comprising:
e) receiving at least one neural signal associated with a time point; and
f) determining, based on the subset, at least one simulated behavior associated with the at least one neural signal.

3. The method of claim 1 or 2, wherein (c) comprises: (i) determining a mapping matrix between the plurality of neural time series signals and the at least one behavioral time signal; (ii) determining a correlation matrix between each pair of neural time series signals and between each neural time series signal and the at least one behavioral time signal; and (iii) subtracting the correlation matrix from the mapping matrix to thereby form a predictability matrix.

4. The method of claim 3, wherein the mapping matrix comprises a convergent cross mapping (CCM) matrix.

5. The method of claim 3 or 4, wherein the correlation matrix comprises a linear correlation matrix.

6. The method of any one of claims 3-5, wherein the correlation matrix comprises a Pearson correlation matrix.

7. The method of any one of claims 3-6, wherein (d) comprises: (i) determining an acyclic directed graph based on the predictability matrix; and (ii) determining, from the acyclic directed graph, the subset.

8. The method of any one of claims 2-7, wherein (f) comprises: (i) determining, based on the subset, a plurality of simulated neural signals associated with a plurality of simulated time points, each simulated time point occurring at a later time than the time point; (ii) determining a geometric weighting between the plurality of simulated neural signals; and (iii) determining, based on the geometric weighting, the at least one simulated behavior.

9. The method of claim 8, wherein the geometric weighting comprises a simplex weighting.

10. The method of claim 8 or 9, further comprising repeating (i)-(iii) to determine a plurality of simulated behaviors.

11. The method of any one of claims 1-10, further comprising measuring the plurality of neural time series signals and the at least one behavioral time series signal.

12. The method of any one of claims 2-11, further comprising measuring the at least one neural signal associated with the time point.

13. The method of any one of claims 1-12, wherein each neural time series signal is selected from the group consisting of: an evoked potential signal, a far-field evoked potential signal, a near-field evoked potential signal, a single-neuron extracellular signal, a multi-neuron extracellular signal, a microelectrode signal, a microelectrode array signal, a sharp electrode signal, a patch-clamp electrode signal, an optical signal, a fluorescence signal, an intrinsic optical change signal, an electroencephalography (EEG) signal, a magnetoencephalography (MEG) signal, a magnetic resonance imaging (MRI) signal, and a functional MRI (fMRI) signal.

14. A system comprising:
a non-transitory memory; and
one or more processors coupled to the non-transitory memory and configured to read instructions from the non-transitory memory to cause the system to perform operations comprising:
a) receiving a plurality of neural time series signals, each neural time series signal associated with at least one neural signal resulting from at least one location in a brain of a subject;
b) receiving at least one behavioral time series signal, the behavioral time series signal associated with at least one behavior of the subject;
c) determining a plurality of causality measures between each pair of neural time series signals and between each neural time series signal and the at least one behavioral time series signal; and
d) determining, from the plurality of causality measures, a subset of the plurality of neural time series signals that correlate with the at least one behavior.

15. The system of claim 14, wherein the operations further comprise:
e) receiving at least one neural signal associated with a time point; and
f) determining, based on the subset, at least one simulated behavior associated with the at least one neural signal.

16. The system of claim 14 or 15, wherein (c) comprises: (i) determining a mapping matrix between the plurality of neural time series signals and the at least one behavioral time signal; (ii) determining a correlation matrix between each pair of neural time series signals and between each neural time series signal and the at least one behavioral time signal; and (iii) subtracting the correlation matrix from the mapping matrix to thereby form a predictability matrix.

17. The system of claim 16, wherein the mapping matrix comprises a convergent cross mapping (CCM) matrix.

18. The system of claim 16 or 17, wherein the correlation matrix comprises a linear correlation matrix.

19. The system of any one of claims 16-18, wherein the correlation matrix comprises a Pearson correlation matrix.

20. The system of any one of claims 16-19, wherein (d) comprises: (i) determining an acyclic directed graph based on the predictability matrix; and (ii) determining, from the acyclic directed graph, the subset.

21. The system of any one of claims 15-20, wherein (f) comprises: (i) determining, based on the subset, a plurality of simulated neural signals associated with a plurality of simulated time points, each simulated time point occurring at a later time than the time point; (ii) determining a geometric weighting between the plurality of simulated neural signals; and (iii) determining, based on the geometric weighting, the at least one simulated behavior.

22. The system of claim 21, wherein the geometric weighting comprises a simplex weighting.

23. The system of claim 21 or 22, wherein the operations further comprise repeating (i)-(iii) to determine a plurality of simulated behaviors.

24. The system of any one of claims 13-23, wherein each neural time series signal is selected from the group consisting of: an evoked potential signal, a far-field evoked potential signal, a near-field evoked potential signal, a single-neuron extracellular signal, a multi-neuron extracellular signal, a microelectrode signal, a microelectrode array signal, a sharp electrode signal, a patch-clamp electrode signal, an optical signal, a fluorescence signal, an intrinsic optical change signal, an electroencephalography (EEG) signal, a magnetoencephalography (MEG) signal, a magnetic resonance imaging (MRI) signal, and a functional MRI (fMRI) signal.
